(19) United States
(12) Patent Application Publication
(10) Pub. No.: US 2026/0120366 A1
(43) Pub. Date: Apr. 30, 2026

(54) METHOD FOR GENERATING AN AUGMENTED IMAGE REPRESENTATION BY A MEDICAL VISUALIZATION SYSTEM, AND MEDICAL VISUALIZATION SYSTEM

(71) Applicant: Carl Zeiss Meditec AG, Jena (DE)

(72) Inventors: Matthias Hillenbrand, Oberkochen (DE); Markus Philipp, Oberkochen (DE)

(21) Appl. No.: 19/373,464

(22) Filed: Oct. 29, 2025

(30) Foreign Application Priority Data
Oct. 29, 2024 (DE) 10 2024 131 547.4

(51) Int. Cl. G06T 11/60; A61B 90/20; G06T 5/70; G06T 7/70; G16H 30/40

(52) U.S. Cl. CPC G06T 11/60; A61B 90/20; G06T 5/70; G06T 7/70; G16H 30/40; G06T 2207/10012; G06T 2207/10056; G06T 2207/20221; G06T 2207/30004; G06T 2210/22

(57) ABSTRACT

A method for generating an augmented image representation by a medical visualization system is provided. The medical visualization system includes at least one image capture device configured for microscopic imaging of an examination region. The method includes receiving a trigger signal, providing topography information when the trigger signal has been received, the topography information representing a surface to be captured, with the surface to be captured being at least partially arranged in the examination region, generating the augmented image representation by overlaying additional information on at least one section of a microscopic image representation while taking the topography information into account. In addition, a medical visualization system is provided.

### CROSS REFERENCE TO RELATED APPLICATIONS

This application claims priority to German patent application DE 10 2024 131 547.4, filed Oct. 29, 2024, the entire content of which is incorporated herein by reference.

### TECHNICAL FIELD

The disclosure relates to a method for generating augmented image representation by a medical visualization system and to a medical visualization system.

### BACKGROUND

Surgical microscopes, inter alia, are used to prepare and perform medical operations on a patient in particular. During a treatment, such surgical microscopes are used by a user, e.g., a surgeon or an assistant, to provide a representation, in particular a magnified representation, of an examination region, in particular in or on the situs of the patient. To this end, a surgical microscope may include an objective or an objective system for generating a real optical image representation of the examination region. The objective may include optical elements for beam guidance and/or beam shaping and/or beam steering. In particular, an optical element may be a lens.

Surgical microscopes find use in medical facilities, but also in laboratories or in industrial applications. Exemplary medical fields of application are neurosurgery, eye surgery, ENT surgery, plastic or reconstructive surgery and orthopedic surgery. This list is not exhaustive. In general, they may be used in all medical applications, e.g., in the field of surgery, which require a magnified and high-resolution view of an examination region, e.g., the operating field, in particular in order to be able to perform precise interventions.

A distinction can be made between analog and digital surgical microscopes. In contrast with digital surgical microscopes, analog surgical microscopes do not capture image representations, which are subsequently displayed on, e.g., a screen for the magnified representation of the examination region, but instead offer a directly visually capturable magnification of the examination region for the user. In this context, radiation reflected or scattered off the examination region reaches through the objective into at least one beam path and to at least one output portion, through or into which the user gazes, in order to visually capture the radiation and hence also the generally magnified representation of the examination region. An exemplary embodiment of an output portion is a so-called eyepiece, into or through which the user gazes in order to optically capture the examination region with at least one eye.

Digital surgical microscopes include exactly or at least one image capture device for microscopic imaging, which captures radiation in a beam path of the surgical microscope in order to generate an image representation, in particular a magnified image representation, wherein this image representation may be displayed to the user or else to multiple users on one or more display device(s). Such an image representation is also referred to as microscopic image representation hereinafter. Consequently, a highly resolved visualization may be rendered possible. The image representation may be generated in the form of an image signal, in particular a transmittable image signal, which encodes or represents the image representation. In that case, an image signal may be transmitted in the form of a data signal, in particular in a wired or wireless fashion. In contrast with analog surgical microscopes, purely digital surgical microscopes do not include any output portion for visually capturable radiation, i.e., do not include an eyepiece in particular.

Digital surgical microscopes allow the recording of image representations and videos, and also the storing and further processing thereof. By applying image processing methods, it is possible in particular to adapt contrast, brightness and other parameters in order to optimize an image quality of the image representations generated. Hybrid surgical microscopes may include both at least one image capture device and at least one output portion. For example, the radiation guided in a beam path of the surgical microscope may be split using a beam splitter, with a first component being guided to the output portion and a further component being captured by the at least one image capture device.

Stereo surgical microscopes that generally include two separate beam paths for beam guidance and provide the user with a depth impression of the examination region are also known. To this end, the beams guided in the two beam paths may be visually captured by the user via output portions. In an alternative to that or in addition, digital surgical microscopes include two image capture devices which each capture the beams in one of the beam paths in order to generate an image representation, wherein the user is then provided with a three-dimensional image representation via a suitable display device on the basis of the two image representations, which may also be referred to as corresponding image representations. In this case, the image capture devices form parts of a stereo (camera) system.

The surgical microscope may form a medical visualization system, or the medical visualization system may include the surgical microscope. Constituent parts of the medical visualization system explained hereinafter may be constituent parts of the surgical microscope or constituent parts that are formed differently from the surgical microscope.

The provision of an augmented representation of an examination region for a user is also known. An augmented representation may be a representation of the real examination region which is augmented in computer-assisted fashion, in particular by virtue of at least one virtual object and/or other additional information, as additional information, being superimposed or overlaid on the representation of the real examination region. The augmented representation may be displayed to a user in the form of an augmented image representation on a display device or may be provided in a manner that can be captured visually by way of an output portion.

Additional information may be provided in the form of data that represent or encode a geometric description of a space, in particular a three-dimensional space, especially with objects arranged therein. Additional information may also be information generated from such data, for example information generated by rendering. Rendering or image synthesis is understood to mean the process of the computer-implemented generation of a photorealistic or non-photorealistic image from a 2-D or 3-D model. Multiple models may be defined in a scene file, which contains objects in a defined language or data structure. The scene file may contain information relating to the geometry, point of view, texture, illumination and shadowing, which describes the virtual scene. The data contained in the scene file are then transmitted to a rendering program, which processes them and outputs them in a digital image or raster graphics file, or which generates such a file. A software application or component that performs the rendering is referred to as a rendering engine, rendering system, graphics engine or simply as a renderer.

An important requirement within the scope of augmentation is that an observer of the augmented image representation, in particular a surgeon, is not disturbed or distracted by the augmentation during their activity. In particular, it is desirable that surgeons may perform surgery ergonomically even if they observe an examination region with an overlaid augmentation. In particular, it is important that an object, e.g., a tumor, situated spatially behind a surface of the examination region in a viewing or observation direction is also represented in such a way in an augmented image representation that it is not incorrectly located spatially in front of the surface and possibly even superimposed on objects arranged there, since this may make the visual perception more difficult for the observer. A stereo depth impression during an observation through a stereo surgical microscope may also be falsified thereby. This may especially be the case when even augmented objects are superimposed in stereoscopically perceivable fashion since the corresponding three-dimensional perception may be confusing to the observer.

If further objects, e.g., surgical instruments, are in a field of view of the medical visualization system, the augmentation may be problematic if it represents objects or parts thereof that are located above the surface of the examination region in the viewing direction, with these objects in reality only being arranged below this surface. An example in this respect is the complete representation of a tumor object, in which portions already removed in reality and located above a surface of the examination region in the viewing direction are represented. In such a case, an observer may be confused when perceiving information regarding a relative position between the object and the examination region.

Usually, a spatial position of a surface of the situs is detected and what is known as clipping is performed on the basis thereof in order to remove that portion of the objects that is located above the surface of the examination region in the viewing direction. However, an inaccurate detection of the spatial position may lead to an inaccurate augmentation, e.g., if the detection is influenced by artifacts. A concealment of the surface, e.g., by instruments, may also lead to an inaccurate augmentation.

US2010/295931 A1 relates to the technical field of medical navigation image output. The medical navigation is used within the scope of image-controlled surgery and assists the surgeon with optimal positioning of their instruments, with reference being made to, e.g., previously acquired image data of the patient. Hence, the treating physician has an image output, e.g. a monitor, available, on which they may recognize where their instrument or their functional part is situated in relation to specific body regions of the patient.

US 2015/221105 A1 discloses imaging systems, imaging apparatuses and imaging methods, which fuse parts of a multidimensional reconstructed image with multidimensional visualizations of at least one part of an operating site. The imaging systems may generate multidimensional reconstructed images on the basis of preoperative image data. The imaging systems may display a part of the multidimensional reconstructed image in a selected portion of the visualization.

### SUMMARY

Hence, it is an object of the disclosure to provide a method for generating an augmented image representation by a medical visualization system and a medical visualization system, which computationally efficiently increase a representation quality of the augmented image representation for improved perception of an imaged examination region given an augmentation and which overcome at least one of the disadvantages explained above, in particular ensure that objects no longer present in reality, or parts thereof, are not incorrectly displayed with the augmented image representation.

The object is achieved by a method for generating an augmented image representation by a medical visualization system as described herein.

The medical visualization system includes at least one image capture device for microscopic imaging of an examination region.

In particular, the medical visualization system includes a surgical microscope or may be formed by the surgical microscope. Surgical microscopes and their technical features were already briefly explained at the outset. In particular, the surgical microscope may be a stereomicroscope. The surgical microscope may also be embodied as an endoscope.

The surgical microscope may include a microscope body. The objective explained at the outset may be integrated into the microscope body or secured, in particular releasably secured, to the latter. In this case, the objective may be arranged in a fixed position relative to the microscope body. In addition to the objective, the microscope body may likewise include or form at least one beam path for the microscopic imaging and/or further optical elements for beam guidance and/or beam shaping and/or beam deflection. In the case of analog and hybrid surgical microscopes, the microscope body may include at least one securing interface for securing, in particular releasably securing, an output element, e.g., an eyepiece. The microscope body may include or form a housing or be arranged in a housing. Constituent parts of the surgical microscope, e.g., an image capture device for microscopic imaging, may be arranged in or on the housing.

In addition to the surgical microscope, the medical visualization system may include a stand for mounting the surgical microscope. The surgical microscope, in particular the microscope body, may be mechanically secured to the stand. The stand is configured such that it allows a movement of the surgical microscope in space, in particular with at least one degree of freedom, typically with six degrees of freedom, wherein a degree of freedom may be a translational or a rotational degree of freedom. Moreover, the stand may include at least one drive device for moving the surgical microscope. Such a drive device may be a servo motor, for example. Of course, the stand may also include means for transmitting forces/moments, for example gear units. In particular, it is possible for the at least one drive device to be driven such that the surgical microscope carries out a desired movement and thus a desired change in pose in space or adopts a desired pose, i.e., a position and/or orientation, in space. For example, the at least one drive device may be driven such that an optical axis of an objective of the surgical microscope adopts a desired orientation. Furthermore, the at least one drive device may be driven such that a reference point of the surgical microscope, for example a focal point, is positioned at a desired position in space. A target pose may be specified by a user or by another superordinate system. Methods for controlling the at least one drive device on the basis of a target pose and a kinematic structure of the stand are known to a person skilled in the art.

Furthermore, the medical visualization system may include a display device or possibly even multiple display devices for displaying the image representations. The display device may serve to display two-dimensional or three-dimensional image representations. In particular, a three-dimensional image representation may be or include a stereo image pair, wherein the image representations of this image pair are stereoscopic image representations. Typical display devices are screens, in particular 3-D screens, head-mounted displays (HMD) or digital eyepieces, which may also be referred to as so-called booms.

Furthermore, the medical visualization system, in particular the surgical microscope, may include one or more of the elements listed below:
at least one white-light illumination device,
at least one infrared illumination device,
at least one fluorescence illumination device for exciting fluorescence,
at least one beam filter for providing excitation radiation with wavelengths from a broader spectrum, e.g., the spectrum of the white-light illumination device,
at least one fluorescence detection device for detecting fluorescence,
at least one filter device for filtering radiation from a broader spectrum, e.g., for capture by an image capture device for microscopic imaging,
at least one image capture device of an optical pose detection device, which may also be referred to as a surrounding view camera, wherein at least one image representation of the surroundings may be generated using this image capture device,
at least one device for identifying the viewing direction,
at least one pose detection device for determining a pose, i.e., a position and/orientation, of at least the surgical microscope,
at least one input device for operating the visualization system,
at least one interface for data transmission to or from a further system or a further device,
at least one device for determining depth information, in particular in relation to the elements arranged in the capture region of the surgical microscope and possibly embodied as a distance measuring device or sensor for example, and
at least one storage device for storing signals and/or information, in particular in retrievable fashion.

An image capture device may include a complementary metal-oxide-semiconductor (CMOS) or a charge-coupled device (CCD) sensor in particular. A capture region of the surrounding view camera may include the capture region of the surgical microscope in full or at least in part. Alternatively, a capture region of the surgical microscope may include the capture region of the surrounding view camera in full or at least in part.

In a fluorescence visualization mode, a filter device may be introduced into an observation beam path, whereby a representation of the examination region that is filtered by the filter device is made available to an observer. This radiation may be detected by the at least one image capture device for microscopic imaging. Alternatively, fluorescence radiation may also be detected by a capture device that differs from the image capture device for microscopic imaging, e.g., by a spectral camera. The fluorescence mode advantageously allows intraoperative tissue differentiation. Tumor tissue may be visualized using fluorescence-based image representations. Nerve tissue may be visualized using polarization-contrast-based image representations.

Medical visualization systems may be operated, e.g., by way of a manual operation of a constituent part, in particular the surgical microscope, or of a corresponding input device, by way of voice control, gesture control, gaze control, image-based control or other operating methods. The medical visualization system or the surgical microscope may include the devices needed to this end. An image-based control may include, in particular, the generation of operating or control signals by evaluating at least one image representation that was generated by an image capture device for microscopic imaging or by an image capture device of an optical pose detection device.

Adjustable operating parameters of the medical visualization system or of the surgical microscope may be formed by one or more of the parameters listed below:
magnification factor or zoom factor,
working distance or focal position,
capture region,
illumination intensity,
illumination spectrum.

The proposed method includes the steps of:
a. receiving a trigger signal, e.g., by way of the interface explained above,
b. providing topography information when the trigger signal has been received, the topography information representing a surface to be captured, with the surface to be captured being at least partially arranged in the examination region, and
c. generating the augmented image representation by overlaying additional information on at least one section of a microscopic image representation while taking the topography information into account.

The trigger signal received in the reception step may be received by way of an interface of the visualization system. It may contain a unique identifier that identifies it as a trigger signal. If the identifier is detected, e.g., by an evaluation device of the visualization system, then the provision step and the generation step may be prompted. In particular, performing the generation step may be prompted in automated fashion once the provision step was performed successfully. In particular, the trigger signal might not be a continuously or periodically generated signal.

The topography information, which is used to generate the augmented image representation in that case, and which may also be referred to as augmentation topography information, may be generated before the provision step or during the provision step by a device for generating topography information. The topography information may be information about a spatial profile and a spatial pose of the surface to be captured. It may be provided in a base coordinate system of the corresponding capture device and then transformed into a reference coordinate system, in particular into a base coordinate system of the additional information, which may also be referred to as world coordinate system, or into a base coordinate system of the visualization system, in particular into a base coordinate system of the stereo system or an image capture device for microscopic imaging. The base coordinate systems may be registered to this end by determining an appropriate transformation rule, and the topography information may be registered information. This is yet to be explained in detail below.

The topography information provided in that case, i.e., the augmentation topography information, may be generated after the generation, reception or detection of the trigger signal and may be triggered by the reception of the trigger signal. However, it is also conceivable that the reception or detection of the trigger signal prompts the provision of the augmentation topography information from topography information that was already generated before the generation, reception or detection of the trigger signal. For example, it is possible that the generation device generates the topography information, which may also be referred to as live topography information, continuously or periodically, and this information is stored in retrievable fashion in a storage device (buffer) of the visualization system. In this case, the generated topography information may be stored with a temporal identifier. Following the reception or detection of a trigger signal, the topography information used to generate the augmented image representation, i.e., the augmentation topography information, may then be provided from the set of the stored topography information, i.e., the stored live topography information, e.g., the most recently generated topography information.

Moreover, at least one signal including the additional information for augmentation may be received before the generation step. This signal, which is subsequently referred to as additional information signal, may be received by way of an interface of the medical visualization system. Exemplary additional information was already presented at the outset. By preference, the additional information signal represents an image representation, in particular a two-dimensional image representation, of the examination region and is provided on the basis of information generated preoperatively or intraoperatively. In this context, the additional information signal may be generated by a further device of the medical visualization system, for example a further image capture device or a sensor. Additionally, the additional information signal may be retrieved from a storage device of the medical visualization system. It is also conceivable that the additional information signal is retrieved from a superordinate system, for example a network. The additional information signal represents or encodes information that is intended to be superimposed on the image representation of the examination region.

The generated augmented image representation may then be transferred, in particular as an image signal, to a display device, with the latter then being controlled to output the image representation in visually perceivable fashion. Visible and/or concealed objects or elements may be imaged in a virtual (3-D) image representation or an augmented (3-D) image representation, in particular, wherein these may also be represented in visually perceivable fashion in that case.

In the case of a stereo surgical microscope, image signals that were generated by the two image capture devices of the stereo system and that represent corresponding image representations of the examination region may be received. In that case, it is possible that an image-representation-specific additional information signal for augmentation is received for each of the corresponding image representations and subsequently used to generate the augmented image representation. For example, an additional information signal that represents a virtual image representation generated from the additional information may be generated by each virtual image capture device. The virtual image capture devices may be optical models of the image capture devices of the stereo system. Hence, an augmentation with a correct perspective is advantageously generated, in particular in a three-dimensional and consistent manner. A virtual image representation may encode a texture, in particular with color and/or transparency information.

To generate the augmented image representation, the additional information may be introduced into, e.g., reflected into, the beam path. For example, it could be projected with a projection device of the surgical microscope onto a projection element, e.g., a panel that is radiation-transmissive arranged in the beam path. In that case, the augmented image representation may be generated by virtue of an image representation being generated on the basis of the beams into which the additional information has been introduced, as explained above. In an alternative, or cumulatively, the radiation that represents the augmented image representation may also be provided by an output portion for visual detection by an observer.

In an alternative, an augmented image representation may be generated by virtue of an image representation of the real examination region being augmented in computer-assisted fashion, in particular by image processing. In this case, the additional information may be overlaid on the image representation of the real examination region. In the case of a stereo surgical microscope, it is possible that two augmented representations are provided for the user. In general, (corresponding) additional information may be introduced into each of the two beam paths of a stereo surgical microscope. In the case of digital stereo surgical microscopes, augmented image representations may in each case be generated from, e.g., image representations generated by the two image capture devices. Hence, an augmented image representation with depth information, i.e., an augmented three-dimensional representation, may also be provided to a user on a corresponding display device or by an output portion.

Additional information presented to a user by augmentation may be information generated preoperatively in particular, which is provided, e.g., in the form of preoperatively generated data, with the latter possibly also serving for treatment planning. Such preoperatively generated data may be volume data in particular. Volume data may be provided in the form of a point cloud, in the form of a voxel-based representation or in the form of a mesh-based representation. In particular, the additional information may also be provided in the form of a signal, in particular a transmittable signal.

Preoperative data may be generated by methods that are based on, e.g., computed tomography or magnetic resonance imaging. Other methods, in particular other imaging methods, such as methods based on ultrasound, x-rays, fluorescence, single photon emission computed tomography (SPECT) or positron emission tomography (PET), may also be used for generation purposes. As a result of such an augmentation, e.g., a tumor object or its contours, which were generated on the basis of preoperative information, may be overlaid on a white-light image.

Various tissue types, e.g., fatty tissue, muscle tissue, tumor tissue but also blood vessels and neural pathways, may be identified in preoperative data generated using a magnetic-resonance-imaging-based method. Bony structures, in particular, may be represented in preoperative data that were generated using a computed-tomography-based method. These tissues, vessels, neural pathways and structures may also be augmented as objects in that case.

In an alternative to the use of preoperatively generated information for the provision of the augmented image representation, or in addition, it is possible that use is made of intraoperative information, i.e., information recorded or generated during the treatment, as additional information for generating the augmented image representation. For example, information may be collected and stored, e.g., during an operation, and may subsequently be used to generate an augmented image representation. This is especially advantageous if there are different visualization modalities that are activated at different times. Using the example of a fluorescence visualization modality, fluorescence information may be presented or overlaid on a white-light image representation.

For augmentation purposes, it is generally necessary to perform a registration between the base coordinate system of the additional information and a base coordinate system of the medical visualization system, in particular of the surgical microscope or an image capture device of the surgical microscope. This registration may be performed prior to augmentation. The registration establishes a spatial relationship of both the additional information and the image representation to a joint base coordinate system, especially also for the information in the image representation generated by the image capture device of the surgical microscope. This joint base coordinate system, also referred to as a reference coordinate system below, may be the base coordinate system of the additional information or the base coordinate system of the medical visualization system, but also a base coordinate system that differs therefrom.

Different methods may be performed for registration purposes, e.g., methods of a model-based registration. In this case, it is possible to detect features in an image representation that correspond to features known in advance, e.g., to geometric features in the additional information, with the registration then being determined on the basis of these corresponding features. For example, the registration may be determined in the form of a transformation matrix, which includes a rotation and/or translation component. An exemplary, model-based registration may be an edge-based registration, wherein the corresponding features are formed for example by a property of at least one edge, typically of multiple edges, in both the image representation and in the additional information. A topography-based registration may also be implemented, especially if a topography can be determined, e.g., using a stereo system of a surgical microscope. Thus, topography information may be determined, e.g., in the at least one image representation, wherein corresponding features or points or portions are then detected both in the additional information and in this topography information and may subsequently be used to determine the registration.

The additional information may thus be registered information. Within the meaning of this disclosure, the “registered” property may mean that a spatial relationship to the reference coordinate system is known, in particular in the form of a transformation matrix. A registered device can generate signals whose spatial relationship to the reference coordinate system is known.

The additional information may be generated by rendering. Thus, a virtual image may be generated by rendering, subsequently be used for augmentation purposes and, e.g., be overlaid on an image representation of the real examination region that was recorded live in particular. The virtual image representation may likewise be provided as an image signal that encodes or represents the virtual image representation.

The virtual image representation may be generated by a virtual image capture device, wherein the latter may be an optical model of an image capture device that can be evaluated mathematically or physically and in particular in computer-assisted fashion. In particular, there may be a computer-implemented calculation of the picture elements of the virtual image representation. This virtual image representation depends inter alia on parameters of the (modeled) image capture device. In particular, the virtual image representation may be generated on the basis of the intrinsic parameters of the image capture device for microscopic imaging, in particular with these parameters. If the corresponding image representations of a virtual stereo system are generated, then these may additionally also be generated on the basis of the extrinsic parameters of the two image capture devices for microscopic imaging with these parameters.

In other words, the parameters of the image capture device(s) of the surgical microscope that serve for microscopic imaging may be taken into account when evaluating the model for generating the virtual image representations. This allows virtual image representations to be generated under the same conditions as real image representations.

Likewise, the virtual image representation may be generated on the basis of a pose, i.e., a position and/orientation, of the (modeled) image capture device of the surgical microscope. In particular, the pose of the image capture device(s) of the surgical microscope that serve for microscopic imaging may be taken into account when evaluating the model for generating the virtual image representations, wherein use can be made of the aforementioned registration information. Taking account of the registration information for example allows determination of the pose of the virtual image capture device in the base coordinate system of the additional information—which may also be referred to as a rendering coordinate system—that corresponds to the real pose of the (modeled) image capture unit of the surgical microscope, and this information may be used for the rendering procedure. In other words, a pose of the at least one virtual image capture device may be the same as the pose of the modeled image capture device in the reference coordinate system. Hence, it is possible to generate a virtual image representation that corresponds to an image representation from the modeled image capture device both in terms of the parameters and in terms of the recording pose. For example, an image representation of the tumor object that should be overlaid may be generated by rendering and subsequently be transmitted as image or video signal and used for augmentation purposes.

Thus, the provision of virtual image representations may require the determination of a current pose of the surgical microscope, in particular of the image capture device. This pose may be determined using a pose detection device. A registration allows determination of a relationship between the base coordinate system of the pose detection device and the base coordinate systems explained above, in particular the base coordinate system of the additional information. This allows the pose of the surgical microscope to be determined in a desired base coordinate system, in particular in the reference coordinate system. Depending on the pose of the surgical microscope, it is possible in turn to determine a pose of the optical axis of the objective or a position of a focal point. Should the surgical microscope be secured to a stand using at least one joint, the pose of the surgical microscope may also be determined depending on a joint position, wherein the joint position may be detected, e.g., by a detection device or a sensor.

The position of the surgical microscope and the additional information may define an above-described scene—i.e., a virtual spatial model - that defines objects and their material properties, light sources and the position and viewing direction of an observer, the surgical microscope in this case.

Self-evidently, it is possible that the pose of at least one further subject or object or a part thereof is also detected by the pose detection device or a further pose detection device. In particular, a subject may be a user of the medical visualization system, e.g., an observer of the examination region or of a display device. For example, it is conceivable to determine a pose of the body part of such a user, e.g., of a hand, an arm or the head. In particular, an object may be a further constituent part of the medical visualization system, in particular a display device. However, an object may also be an item that is not part of the medical visualization system, e.g., an item of equipment such as an operating table or a medical instrument. This allows determination of the pose of the further subject or object in a desired base coordinate system.

Such a pose detection device may also be referred to as tracking system. A tracking system may be a tracking system operating on an optical, electromagnetic or any other basis. The tracking system may be a marker-based tracking system that detects active or passive markers. Markers may be arranged on objects or subjects whose pose should be detected by the tracking system. In particular, an optical tracking system may include optically detectable markers. In particular, an optical tracking system may be a system for monoscopic pose detection. In this case, the pose of an object may be determined by evaluating a two-dimensional image representation, in particular exactly one two-dimensional image representation. In particular, intensity values of pixels (picture elements) of the two-dimensional image representation may be evaluated in order to determine the pose.

It is further conceivable that the medical visualization system includes at least one image capture device of an optical pose detection device that may be a constituent part of the surgical microscope in particular. The latter may also be referred to as surrounding view camera and in particular serve for monoscopic pose detection. Hence, the surrounding view camera may be part of an optical tracking system.

A tracking system may also be part of an input device, wherein, e.g., a gesture control or a viewing direction control is implemented on the basis of information generated by the tracking system.

The fact that the augmented image representation is generated while taking the topography information into account may mean that the additional information used for the augmentation is determined on the basis of the topography information. In the simplest of examples, what is known as clipping may be performed on the basis of the topography information. By clipping, the additional information to be augmented may be determined from the (additional information not adapted by clipping), in such a way that only the objects located spatially behind the surface of the examination region in a viewing or observation direction of the visualization system are imaged by the augmented image representation. Other exemplary ways of generating the augmented image representation in this way will still be explained below.

In summary, the topography information used to generate the augmented image representation is provided when a trigger signal has been received. It is also possible that the reception of the trigger signal triggers the generation and optional display of the augmented image representation. This may also be referred to as updating the augmented image representation.

In other words, receiving the trigger signal may ensure that topography information is only generated or provided and used to update an augmented image representation when this is expedient and/or necessary. In particular, there need not be any continuous or periodic generation or provision of the topography information and updating of the augmented image representation.

The method according to the disclosure advantageously enables an improved representation quality for an examination region represented by an augmented image representation since a contradictory perception of information by an observer is reliably avoided by taking topography information, in particular current topography information, into account. The trigger-signal-dependent provision of the topography information advantageously yields this in computationally efficient fashion, in particular since this makes it possible to avoid a continuous or periodic provision of topography information or hence a continuous or periodic generation of the augmented image representation, which may be triggered by the provision of the topography information, while taking this topography information into account.

In a further exemplary embodiment, the topography information, i.e., the augmentation topography information explained above, is generated and provided following the reception of the trigger signal. This advantageously yields a particularly efficient method, especially since there is no need to generate any topography information that is then ultimately not taken into account when generating the augmented image representation. Alternatively, the topography information, i.e., the augmentation topography information explained above, is provided from already generated topography information, in particular the likewise explained live topography information. This has already been explained above. Advantageously, already generated topography information may be used as a result, in order to take this into account when generating the augmented image representation. Thus, topography information generated for other purposes, e.g., in the topography-based registration explained above, may be used efficiently.

In a further exemplary embodiment, the topography information is generated by evaluating microscopic image representations that are generated by the at least one image capture device for microscopic imaging of the medical visualization system. In particular, there may be a stereogrammetric generation of the topography information, with the three-dimensional topography information being generated from two-dimensional microscopic image representations in particular. In the case of a stereo system, the image representations generated by the two image capture devices are typically used to this end. In other words, the (corresponding) microscopic image representations generated by the image capture devices of a stereo system may be used to generate the topography information.

In an alternativ, or cumulatively, this topography information is generated by evaluating output signals from a distance measuring device, in particular a TOF or lidar device. In particular, the distance values generated by such a device may be used to generate the topography information.

To generate the topography information, a relative movement may be implemented between the visualization system or a part thereof and the examination region, typically by moving the visualization system or a movable part of this system, with the examination region being arranged stationarily. However, it is also possible to arrange the visualization system stationarily and move the examination region or to move both the visualization system and the examination region.

This advantageously yields a reliable and accurate provision of topography information, in particular using components that optionally already are constituent parts of the visualization system. This in turn improves the quality of the augmented image representation.

In a further exemplary embodiment, the trigger signal is generated when a trigger movement is detected. In particular, methods for gesture control may be used to generate the trigger signal. The trigger movement may also be detected using a pose detection device or a tracking system, in particular by evaluating (sequences of) microscopic image representations and/or image representations of the surroundings and/or topography information. In particular, the trigger movement may be performed by the hand or the arm of a user or using an instrument operated by the user in particular. In particular, the instrument may include a unique identity, e.g., provided by a (unique) marker arrangement or a (unique) marker pattern. In that case, a trigger movement may only be carried out by an instrument with a predetermined identity. Thus, in this case, the trigger signal is only generated when an instrument with a predetermined identity is detected, and this instrument carries out the trigger movement. The trigger movement may be a predetermined movement, in particular a movement with predetermined properties. Thus, movements may be captured, and properties of the movement or of parts thereof may be determined. The trigger signal may be generated if the properties correspond to the predetermined properties. This advantageously yields a contactless generation of a trigger signal.

In an alternative, or cumulatively, the trigger signal may be generated by virtue of an input device being operated, especially in a predetermined manner. This input device may be operated by a user, e.g., using a hand or a foot. Thus, an input device may be, e.g., a foot switch or a hand switch. An input device may also take the form of a human-machine interface. An input device may also include a keyboard, a mouse or a touchscreen. Further corresponding input devices were explained above. This advantageously yields a particularly reliable generation of a trigger signal.

In a further alternative, or cumulatively, the trigger signal may be generated when a predetermined change is detected in microscopic image representations and/or image representations of the surroundings. In a further alternative, or cumulatively, the trigger signal may be generated when a predetermined change is detected in the topography information.

To this end, e.g., image representations or pieces of topography information generated at different times may be compared, wherein these are generated, e.g., continuously or periodically and stored as explained above. In that case, the trigger signal may be generated when, e.g., a measure that represents a deviation between the various image representations or pieces of topography information is greater than a predetermined threshold value. To this end, the comparison may also be performed continuously or periodically.

In the case of a comparison of pieces of topography information, the measure may be, e.g., a mean distance between corresponding points or sections of the surface represented by the corresponding pieces of topography information. In the case of a comparison of image representations, the measure may be, e.g., a measure for a lack of similarity between images.

In particular, it may be possible that the predetermined change is caused by what is known as a brain shift, wherein a change in the topography caused by this brain shift is advantageously recognized automatically, and an update of the augmented image representation may also be generated automatically.

It may also be possible that the predetermined change is caused by a predetermined object movement, e.g., if a previously imaged hand or a previously imaged instrument is no longer imaged by the image representations or the topography information. Hence, a change in the captured topography caused by this object movement may advantageously be recognized automatically, and an update of the augmented image representation may also be generated automatically.

It is also possible to determine a periodicity or a measure for the periodicity of the changes. It is conceivable that only those changes whose periodicity is longer than a predetermined periodicity lead to the generation of a trigger signal. Hence, it is possible e.g. to avoid that pulse-related changes in the topography lead to the generation of a trigger signal.

It is possible that the measure is determined only on the basis of non-falsified shares of the topography information, which are explained in detail below, or on the basis of image sections in which the surface is imaged.

This advantageously yields a generation of the trigger signal that is adapted to actual changes in the topography and hence efficient, and this also applies to the linked provision of the topography information and the generation of the augmented image representation. In particular, a trigger signal is only generated should the topography have changed by more than a predetermined measure and the previously used topography information would falsify an augmented image representation, optionally by more than a predetermined measure.

In a further exemplary embodiment, the topography information is provided following the reception of the trigger signal and/or the topography information is provided to detect a predetermined change, by virtue of generated topography information being processed to reduce falsifying information. For example, falsifying information may represent objects or structures or parts thereof that are not part of the actual topography but were imaged therein when the topography information was generated. For example, such an object might be a swab that is arranged on the surface to be captured. Such an object might also be an instrument that conceals the surface to be captured from the view of the device for acquiring the topography information and that is imaged in the topography information when the latter is generated, in particular in the case of a stereogrammetric generation of the topography information, more particularly when microscopic image representations are used.

In order to reduce these falsifying information components, the falsifying information components that do not represent the actual topography and optionally also the non-falsifying information components that represent the actual topography may be identified in the topography information. To this end, especially the output signals, which may be image signals in particular, from the device for generating topography information may be processed. For example, the falsifying information components may be identified using a segmentation method. Hence, it is possible in particular to identify information components that are not part of the actual topography, e.g., information components representing instruments and tools.

However, in order to identify objects or structures or parts thereof that are not part of the actual topography, it is also possible that output signals from a device that differs from the device for generating the topography information are processed. Then, the falsifying information components may be determined on the basis of the objects or structures identified thus, especially when the base coordinate systems of the devices are registered. Thus, e.g., such objects or structures may be identified in the microscopic image representation or an image representation of the surroundings.

Furthermore, the falsifying information components may be removed and optionally replaced, e.g., by replacement information. Such replacement information may be generated, e.g., from predetermined topography information. In an alternative, replacement information may be determined on the basis of the non-falsified information components. However, it is also possible that the falsifying information components are not taken into account when generating the augmented image representation.

In an alternative, or cumulatively, the aforementioned topography information is provided by virtue of topography information generated at different times being fused. For example, topography information generated at different times could be averaged in order to then provide the topography information used to generate the augmented image representation, i.e., the resultant topography information. The resultant topography information could also be combined from pieces of information generated at different times. This advantageously yields a provision of accurate topography information.

In a further exemplary embodiment, fused topography information is smoothed topography information. For example, smoothed topography information may be generated by virtue of a sliding mean or a sliding median being determined on the basis of at least two pieces of topography information, albeit typically by more than two pieces of topography information, generated at different times, or by virtue of exponential smoothing being performed. To this end, it is possible to use topography information that was generated with a predetermined and in particular large time constant. Hence, such topography information may also be referred to as temporally smoothed topography information. However, it is also conceivable that the topography information is determined as spatially smoothed topography information.

In an alternative, or cumulatively, different sections of the surface represented by the fused topography information are represented by topography information generated at different times.

This advantageously yields a provision of topography information that is as accurate as possible and can be obtained in an easily implementable manner.

In a further exemplary embodiment, the fused topography information is generated by stitching the topography information generated at different times. Stitching denotes a process in which information components of the topography information generated at different times are combined, in particular seamlessly, to form resultant topography information. In particular, this may be implemented in such a way that the surface arranged in the acquisition region of the device for generating topography information is represented in full or to more than a predetermined measure by this resultant topography information. This advantageously also yields a provision of topography information that is as accurate as possible.

In a further exemplary embodiment, falsifying information or falsifying information components is/are replaced by topography information generated at a different time. This was already explained above and hence enables a determination of topography information that is as accurate as possible, even if foreign objects are possibly present in the acquisition region of the device for generating the topography information. In particular, a segmentation method may be used to identify those information components in the topography information that represent falsifying tools or instruments, and these information components may subsequently be replaced by non-falsifying information components from topography information generated at a different time. The non-falsified information components represent that part of the actual topography that is represented in falsified fashion in the falsified information components. In this context, the replacement may be implemented by the aforementioned stitching. This advantageously also yields a provision of topography information that is as accurate as possible.

In a further exemplary embodiment, falsifying information represents an object that is not part of the surface to be captured. This and corresponding technical advantages have already been explained above.

In an alternative, or cumulatively, falsifying information represents a foreground region of an image representation of the surface to be captured. To this end, the topography information or the output signals used to determine the topography information, e.g., image representations, may be divided into a foreground region and a background region. This division may also be referred to as segmentation.

If topography information is generated on the basis of image representations, in particular microscopic image representations or image representations of the surroundings, then picture elements or image regions of the image representation may be assigned either to the foreground region or to the background region or may be classified either as foreground or as background. The division may be implemented in such a way that, in particular, elements such as, e.g., objects or structures that should not be overlaid by an augmentation or that are not part of the actual topography are imaged into the foreground region, wherein these elements may be referred to as foreground elements. For example, foreground elements include instruments, especially surgical instruments, hands or fingers or portions thereof. Accordingly, elements that may be overlaid by an augmentation or which are part of the actual topography are imaged in the background region; these elements may also be referred to as background elements. Background elements include tissue in particular. However, background elements may also be instruments. In particular, there may be what are known as hybrid elements, which may be background or foreground elements, depending on the scenario. An exemplary hybrid element could be, e.g., a swab, which is classified as a background element, especially if it is arranged statically and/or without being manipulated in the examination region and/or arranged statically on the surface to be captured. However, the swab may also be classified as a foreground element, especially if it moves by more than a predetermined measure and/or is manipulated by a user. Within the meaning of this disclosure, foreground elements are not restricted to instruments. Furthermore, an object recognition need not necessarily be performed in order to detect foreground elements.

In particular, partial regions of the image representation in which tissue is imaged are classified as parts of the background region.

The division may be implemented, in particular, by virtue of generating an image mask that represents information about the foreground and background regions. Such an image mask is yet to be explained below. The image mask may be represented or encoded by a transmittable signal. Self-evidently, information regarding the division may also be provided in a different way.

In the case of a stereo surgical microscope, the corresponding image representations may each be divided into the foreground region and into the background region.

This advantageously allows a simple identification of falsifying information components in topography information in a current scenario. In contrast to pure object recognition for detecting such falsifying information components, the assignment to the foreground or background region need not be implemented on an object recognition basis. Rather, this allows an object to be assigned to the foreground or background region depending on the scenario and may be accordingly identified as a falsifying information component or as a non-falsifying information component.

In particular, it is possible to receive at least two image signals that were generated successively in time by the at least one image capture device of the surgical microscope and that each represent an image representation of the examination region. Further, it is possible to determine information about a change in the image information over time on the basis of these at least two image representations, and the image representation, in particular the image representation generated at an earlier or later time, is divided into the foreground and background region on the basis of this information. In particular, the change in the image information may be determined on the basis of the change or as the change between the at least two image representations, e.g., as a difference between the image representations or in a manner dependent thereon. The information about a change in the image information over time may typically be information regarding the optical flow that is determinable on the basis of these at least two image representations. In an alternative to determining the information regarding the optical flow, it is also possible to use other methods for determining the information regarding a change in the image information over time, e.g., methods for determining a motion field.

It is also possible to determine depth information for the at least one image representation of the examination region, with the division being implemented on the basis of this depth information. For example, depth information may represent a distance of the element imaged in a picture element from a reference point, a reference line or a reference surface. This distance may be determined in a reference direction. In particular, the distance may be determined in the reference coordinate system. For example, the reference surface may be oriented perpendicular to the optical axis of the surgical microscope, with the reference direction being oriented parallel to the optical axis. For example, a point of intersection between the optical axis and an end glass of the surgical microscope may be arranged in the reference surface.

The depth information may be generated using a device for generating depth information. The latter may be embodied in the same way as the aforementioned device for generating the topography information or as a device that differs therefrom. For example, the device for generating the depth information may be a distance sensor. For example, such a distance sensor may be an OCT distance sensor, a lidar sensor, a time-of-flight sensor or a triangulation sensor such as a structured light sensor. Self-evidently, it is also possible to use distance sensors that generate distance information according to different physical principles of operation. Hence, the topography information may be the same as the depth information, or these pieces of information may be generated on the basis of output signals from the same device. The device for determining the depth information may be registered, wherein the depth information may thus be registered information.

It is also possible to determine reconstruction information, in particular registered reconstruction information, from in particular corresponding image representations of the stereo system; this reconstruction information represents a three-dimensional reconstruction of the capture region, wherein the depth information is determined on the basis of this reconstruction information. For example, it is possible to detect a surface of the examination region in the three-dimensional reconstruction and thereupon determine the distance from the reference point, reference line or reference surface explained above. Furthermore, it is possible to determine depth information from information generated preoperatively. For example, it is possible to detect a skull surface or a dura surface in preoperative information, with in that case, as explained above, it also being possible to determine a distance from the reference point, reference line or reference surface explained above.

In particular, it is possible to generate a depth map for the image representation of the examination region that assigns depth information to each picture element of the image representation. In that case, a picture element whose depth information is greater than a predetermined minimum threshold value and/or less than a predetermined maximum threshold value may be assigned to the foreground region. Otherwise, the picture element may be assigned to the background region. It is also possible to determine a statistical parameter of the depth information generated thus, for example a mean value or a median of all the depth information assigned to the picture elements. Should the depth information assigned to a picture element be greater than or less than the median or the mean value, this picture element may be assigned to the foreground region, otherwise it is assigned to the background region.

It is also possible to determine semantic additional information for the image representation of the examination region or at least a portion thereof, with the division being implemented on the basis of this semantic additional information. The semantic additional information may be information that is taken into account during the division in addition to information that differs therefrom, e.g., in addition to the information—explained above—about a change in the image information over time and/or the depth information. In particular, meaning information may be assigned to the image representation, in particular to a picture element or a set of picture elements, in addition to information required for a visually perceivable representation, for example color and/or transparency information. In particular, the meaning information may be descriptive information regarding the type of element imaged, its relationship to other imaged elements or to the surroundings. This information may be determined in image-based fashion, e.g., using a method for semantic segmentation. Thus, semantic additional information may be information about or from a semantic segmentation of an imaged scene, wherein the segmented scene may be classified into, e.g., image regions, each of which is assigned a class from a set of predetermined classes. For example, the set of predetermined classes may include the classes of “falsifying information component” and/or “non-falsified information component” and/or a further class. In particular, semantic additional information may be information that may be evaluated for the aforementioned division, i.e., division-relevant information.

Additionally, context information may be determined for the image representation of the examination region or for at least a portion thereof, with the division being implemented on the basis of this context information. In particular, context information may be information regarding a spatial or a temporal context. Additionally, context information may be information about a type of current user activity, about a type of a current operating phase or about an operation type. In particular, the context information may differ from the information required for the visually perceivable representation of the image representation. Taking account of context information advantageously yields a very reliable division into foreground and background regions, which in turn improves the representation quality.

Furthermore, the division may be performed with a model, e.g., with a neural network, which was generated by machine learning. In this context, the term machine learning includes or denotes methods for determining the model on the basis of training data. Thus, it is possible to determine the model using methods for supervised learning, with the training data, i.e., a training data record, including input data and output data to this end. In this case, image representations representing the examination region or the corresponding image signals may be provided as input data, with the division of the respective image representation into foreground and background regions being provided as output data.

Input variables for the model-based division may include at least one of the following types of information in addition to the at least one image representation of the examination region or the corresponding image signal:
a) depth information for the at least one image representation of the examination region,
b) at least one image representation of the examination region generated before the image representation of the examination region,
c) an image representation that corresponds to the image representation of the examination region and that was generated by a further image capture device of the surgical microscope,
d) information regarding the classification of imaged objects,
e) information regarding the classification of user activity,
f) information regarding the classification of an operating phase, and
g) information regarding the classification of an operation type.

The types of information according to points d) and e) may form semantic (additional) information. The types of information according to points f) and g) may form context information.

In a further exemplary embodiment, the additional information represents at least one object to be augmented, with only those portions of the object to be augmented that are arranged spatially behind a surface of the examination region in an observation direction of the medical visualization system, in particular of the at least one image capture device for microscopic imaging, being used for the generation of the augmented image representation. In particular, the observation direction may be oriented in particular concentrically with or parallel to an optical axis of the medical visualization system, in particular the optical axis of the at least one image capture device for microscopic imaging or of the stereo system, and may be oriented from the medical visualization system toward the surface.

In this case, the additional information may in particular represent only selected objects or parts of selected objects, in particular pre-selected objects. The selection or pre-selection may be made by a user by operating the aforementioned input device. The (pre-)selection may be automated as well. Additionally, additional information may be generated in such a way that in particular (pre-)selected objects or parts thereof are labeled or presented in accentuated fashion. In particular, labels or accentuation may be labels/accentuation in color. Information regarding a pre-selection may also be stored in a storage device and retrieved from there. In particular, only selected objects may be cropped for augmentation purposes, with non-selected objects not being augmented or not being cropped.

In an alternative, or cumulatively, the generation of the augmented image representation on the basis of both the additional information and the topography information may be implemented in such a way that an object represented by the additional information in the augmented image representation is presented in a manner depending on a distance between the object and the surface, in particular in or parallel to the observation direction.

For example, a degree of transparency used for the representation may be used in distance-dependent fashion. For example, objects or object parts located deeper below the surface may be represented with less transparency in comparison with objects or object parts located not so deep.

An object color or object staining used for representation purposes may also be chosen in distance-dependent fashion. For example, objects or object parts located deeper below the surface may be represented in a different color or with different staining in comparison with objects or object parts located not so deep.

The representation as a labeled or accentuated object explained above may also be implemented in distance-dependent fashion. For example, those objects or parts thereof whose distance from the surface is less than a predetermined measure may be represented in labeled or accentuated fashion. Additionally, objects or object parts located not so deep under the surface may be represented in labeled or accentuated fashion in comparison with objects or object parts located deeper down. Additionally, the type of representation, e.g., the representation of an object using a wireframe representation or the type of the lines used for the representation, may be implemented in distance-dependent fashion.

This advantageously results in a high quality of use and improved applicability of the medical visualization system, which may also mean an improved operation result.

In a further exemplary embodiment, the portions used to generate the augmented image representation to be augmented are generated by cropping the object to be augmented. Parts of a (virtual) object or of a scene that are located in front of the surface in the observation direction explained above, in particular by more than a predetermined measure, may be removed by cropping, which is carried out in computer-implemented fashion in particular. Cropping may be implemented by evaluating a relative pose between the surface represented by the topography information and the objects represented by the additional information. In particular, this relative pose may be determined in the base coordinate system of the stereo system. Cropping allows the implementation of a consistent representation of an augmented image representation, which in turn further improves the quality of use and applicability.

In a further exemplary embodiment, object-specific information is generated on the basis of the topography information, and the augmented image representation is generated in such a way that this object-specific additional information is represented by the augmented image representation in a manner assigned to the object or object portion that is used to generate the augmented image representation. For example, the object-specific information may be information that is or was generated depending on the distance from the surface explained above. For example, the information may be distance information. In this case, the distance from the surface may be represented in the augmented image representation for each object or for (pre-)selected object parts. This allows the quality of use and applicability to be further improved.

In a further exemplary embodiment, the final generation of the augmented image representation is preceded by a preview of the augmented image representation being generated and displayed on a display device, the augmented image representation being determined finally when a preview-specific confirmation signal is generated. This finally determined augmented image representation may then be displayed. The preview-specific confirmation signal may be generated by manipulation or operation of the input device explained above, especially by a user. Should the preview-specific confirmation signal not be generated at all or not be generated within a predetermined period after the start of the representation, the final determination and display cannot be implemented; in particular, the previously used augmented image representation may continue to be used or displayed. However, it is also possible that the preview-specific confirmation signal is generated if no signal for terminating or rejecting the representation is generated within a predetermined period after the start of the representation. This allows the user to check or be able to check the augmented image representation before its final or further use. This advantageously allows the augmented representation to be checked, e.g., for consistency or a representation as desired, whereby the quality of use and applicability are further improved.

In a further exemplary embodiment, the topography information provided is modified dynamically, with the augmented image representation being generated in a manner depending on the dynamically modified topography information. As explained above, changes over time in already generated topography information may be determined, wherein properties of these changes may also be determined in that case. If a first piece of topography information is generated at a first time, it is possible to determine the topography information determined at a comparatively later time by virtue of the first piece of topography information being modified on the basis of these change properties and the time difference between a first time and the later time. In that case, there may also be a renewed or updated generation of an augmented image representation depending on this topography information determined for the later time. In particular, properties of pulse-related changes between the pieces of topography information generated at different times may be determined to this end. In that case, this may also be referred to as pulse-prediction-based determination of topography information or as pulse-prediction-based generation of an augmented image representation. This further improves the quality of use and applicability.

A medical visualization system, in particular including or consisting of a surgical microscope, is also provided. This visualization system includes at least one image capture device for microscopic imaging of an examination region, at least one interface for receiving a trigger signal and at least one evaluation device. The visualization system, in particular the evaluation device, is configured to perform a method according to any of the exemplary embodiments explained in this disclosure.

Furthermore, the visualization system may include at least one interface for receiving an image signal of an image capture device for generating an image representation of an examination region. As explained above, the medical visualization system may in particular include at least one of the following devices:
a device for determining topography information, and
a device for determining portion-specific blurriness and/or shadowing information.

The evaluation device may take the form of or include a computing device. A computing device in turn may include or take the form of at least one microcontroller and/or at least one integrated circuit. In particular, the computing device may generate the virtual picture. It is possible that the evaluation equipment includes at least one graphics processing unit (GPU). The medical visualization system advantageously enables the implementation of a method according to any of the exemplary embodiments described in this disclosure, together with the advantages that have already been explained.

A computer program product having a computer program is also provided, wherein the computer program includes software for carrying out several or all steps, in particular steps b. and c., of the method according to any of the exemplary embodiments described in this disclosure when the computer program is executed by or in a computer or in an automation system. The computer or the automation system may include the evaluation device explained above. The computer program product may in particular include means for performing the rendering, i.e., a rendering engine. The computer program product advantageously enables the implementation of a method according to any of the exemplary embodiments described in this disclosure, together with the advantages that have already been explained.

### BRIEF DESCRIPTION OF THE DRAWINGS

The disclosure will now be described with reference to the drawings wherein:

FIG. 1 shows a schematic flowchart of a method according to an exemplary embodiment of the disclosure,

FIG. 2 shows a schematic flowchart of a method according to a further exemplary embodiment of the disclosure,

FIG. 3 shows a schematic flowchart of a method according to a further exemplary embodiment of the disclosure,

FIG. 4 shows a schematic flowchart of a method according to a further exemplary embodiment of the disclosure,

FIG. 5 shows a schematic flowchart of a method according to a further exemplary embodiment of the disclosure,

FIG. 6A shows a schematic view of an examination region with a surface to be captured,

FIG. 6B shows a visualization of an incorrect augmentation,

FIG. 7A shows a schematic view of an examination region with a surface to be captured,

FIG. 7B shows a visualization of an incorrect augmentation, and

FIG. 8 shows a schematic block diagram of a medical visualization system according to an exemplary embodiment of the disclosure.

### DESCRIPTION OF EXEMPLARY EMBODIMENTS

Identical reference signs hereinafter denote elements having identical or similar technical features.

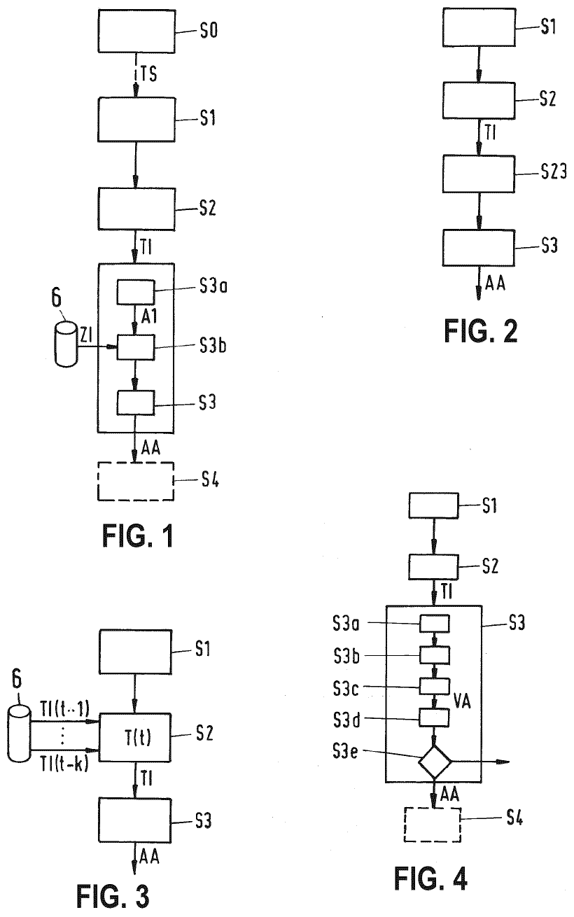

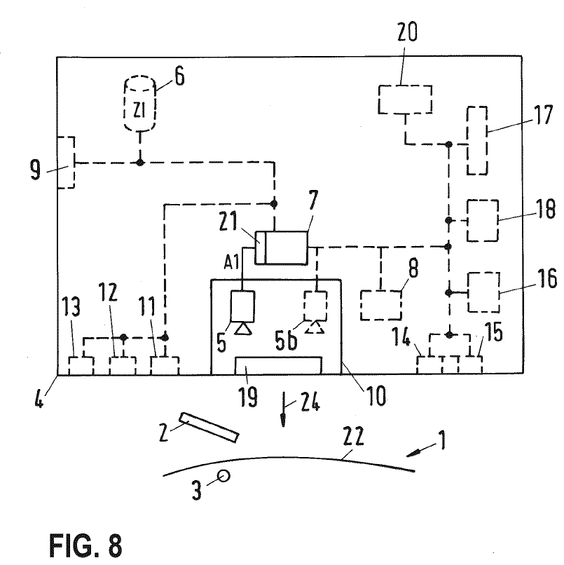

FIG. 1 shows a schematic flowchart of a method according to an exemplary embodiment of the disclosure. A trigger signal TS is received in a reception step S1. This trigger signal TS may have been generated in a generation step S0. For example, the trigger signal TS may have been implemented by operating an input device 18 (see FIG. 8). In an alternative or cumulatively, image representations generated by a surrounding view camera 15 may be evaluated for the detection of a trigger movement, which, e.g., may be carried out by an instrument 2. In an alternative or cumulatively, image representations generated by the image capture device(s) 5, 5b for microscopic imaging, which may also be referred to as microscopic image representations, may be evaluated for the purpose of detecting such a trigger movement.

Additionally, predetermined changes in the image representations generated by the surrounding view camera 15 and/or in the microscopic image representations may be determined. For example, one such predetermined change may be that a currently imaged object, for instance the instrument 2, is no longer imaged or has performed a predetermined object movement. In order to detect such changes, generated image representations may be stored, in particular in a storage device 6 (see FIG. 8) or in a storage device of the medical visualization system 4 that differs therefrom. For example, a picture dissimilarity between these image representations may be determined. The trigger signal TS may be generated once the predetermined change was detected, e.g., if the picture dissimilarity corresponds to or exceeds a predetermined measure.

Topography information TI may also be generated at different times, wherein the trigger signal TS is generated if predetermined changes are detected in this topography information TI. The pieces of topography information TI generated at different times may be generated periodically in particular and may be stored in a storage device 6 (see FIG. 8) or in a storage device of the medical visualization system 4 that differs therefrom.

This topography information TI may typically be generated by evaluating the microscopic image representations generated by the stereo system, in particular by applying a stereogrammetric method. This topography information TI may also be generated by evaluating output signals of a device 8 for determining depth information in the form of a distance sensor, wherein this device 8 takes the form of, e.g., a lidar or TOF device.

The topography information TI represents a spatial pose of a surface 22, e.g., of a situs in the examination region 1, which is located in the detection region of a device for generating the topography information TI. If further objects, e.g., an instrument 2, are also situated in this detection region in addition to the surface 22, then the topography information TI may be falsified, in particular include a falsified component. It may be possible to identify this falsifying component and a non-falsified component and evaluate only the non-falsified components in respect of the detection of predetermined changes. For example, a mean distance between corresponding points or sections of the surface that are represented by the topography information TI generated at different times may be determined. The trigger signal TS may be generated once the predetermined change was detected, e.g., if the mean distance corresponds to or exceeds a predetermined measure.

The reception of the trigger signal TS is a followed by the provision of topography information TI in a provision step S2. It may be provided by virtue of the topography information TI being generated following the reception of the trigger signal TS, in particular in the manner explained above. In other words, the reception of the trigger signal TS may trigger the generation or acquisition of topography information TI, which is subsequently—as yet to be explained in detail below—taken into account for the generation of the augmented image representation AA. Alternatively, topography information TI may be provided in the provision step S2 from already generated topography information, e.g., by virtue of topography information TI that was previously generated and stored being retrieved.

The augmented image representation AA is generated in a generation step S3 by overlaying additional information ZI on at least one section of a microscopic image representation while taking the topography information TI provided thus into account. In this case, the trigger signal TS may trigger the sequence of provision step S2 and generation step S3. In a first sub-step S3a of the generation step, an image signal may be received, the latter having been generated by at least one image capture unit 5, 5b for microscopic imaging and representing an image representation A1 of the examination region 1.

At least one signal including additional information ZI for the augmentation, which is also referred to as additional information signal, is received in a second sub-step S3b. Such additional information ZI may represent or encode a geometric description of a space, in particular a three-dimensional space, in particular with objects such as the tumor object 3 depicted in FIG. 11 arranged therein.

FIG. 1 illustrates that the additional information signal is stored in retrievable fashion in a storage device 6 which may be part of the medical visualization system 4. However, it is also possible that the additional information ZI is acquired intraoperatively and/or retrieved from a network via a suitable interface. In particular, the additional information signal may also represent an image representation, typically an image representation that has the same size as the image representation A1 generated by the image capture device 5. The image representation represented by the additional information signal may in particular be a virtual image representation which is generated from additional information ZI generated preoperatively. As already explained at the outset, such a virtual image representation may be generated using a virtual image capture device which is an optical model of the image capture device 5.

The additional information ZI is processed depending on the topography information TI in a further sub-step S3c. For example, the additional information ZI may be processed in such a way that only those sections of an object to be augmented—e.g., of the tumor object 3—that are arranged spatially behind the surface 22 of the examination region 1 in an observation direction of the medical visualization system 4 are used to generate the augmented image representation AA. To this end, clipping may be performed in the further sub-step S3c, wherein objects or parts thereof represented by the additional information ZI that are arranged spatially in front of the surface 22 of the examination region 1 in an observation direction of the medical visualization system 4 are cropped by this processing and are not used to generate the augmented image representation AA.

It is possible that the additional information ZI used (processed) for augmentation purposes represents only selected objects or parts of selected objects, in particular pre-selected objects. The selection or pre-selection may be made by a user, in particular by operating the aforementioned input device 18. Additionally, additional information ZI may be generated in such a way that in particular (pre-)selected objects or parts thereof are represented with a colored label. In addition to that or in an alternative, the additional information ZI may be processed in this way. Furthermore, processing may be such that an object represented by the processed additional information ZI is represented depending on a distance between the object and the surface 22, in particular in or parallel to the observation direction, e.g., in the form of a distance-dependent degree of transparency or a distance-dependent colored label.

In that case, the augmented image representation AA is generated by virtue of the processed additional information ZI being overlaid on the image representation A1 or a part thereof.

In an optional display step S4, the augmented image representation AA generated thus may be displayed, e.g., on a display device 20 of the medical visualization system 4.

The sequence of steps S1, . . . , S4 depicted in FIG. 1, in particular the sequence of sub-steps S3a, S3b, S3c, is not a mandatory sequence in this case. For example, the first sub-step S3a and the second sub-step S3b may be carried out before the reception step S1 or before the determination step S2.

FIG. 2 shows a schematic flowchart of a section of a method according to a further exemplary embodiment of the disclosure. In contrast to the exemplary embodiment depicted in FIG. 1, the topography information TI generated or provided in the provision step S2 is processed for the purpose of reducing falsifying information. This is implemented in a processing step S23, which is carried out after the provision step S2 but before the generation step S3 in the illustrated exemplary embodiment.

As an alternative, or in addition, the topography information TI generated in the generation step S0 may also be processed to reduce falsifying information or falsifying information components. Falsifying information components may be caused, e.g., by (foreign) objects in the detection region of a device for generating topography information TI, e.g., by an instrument 2 (see FIG. 11). In other words, such information components may represent not a section of the surface 22 but a foreign object that is not part of the surface 22 to be captured. For example, such a foreign object may be an instrument 2 (see FIG. 11).

However, such falsifying information may also represent a foreground region of an image representation, in particular a microscopic image representation, of the surface 22.

Such falsifying information components may be identified in a first sub-step of the processing step S23. For example, a segmentation method may be applied in order to detect information components caused by foreign objects.

Additionally, the topography information or the output signals used to determine the topography information, e.g., image representations, may be divided into a foreground region and a background region. This division may also be referred to as segmentation. If topography information is generated on the basis of image representations, in particular microscopic image representations or image representations of the surroundings, then the division may be implemented by virtue of an image mask being generated, the latter representing information about foreground and background regions. Such an image mask may be provided as an image representation in particular, more particularly as a two-dimensional image representation, wherein an image size of the image mask may correspond to an image size of the image representation used to generate topography information. In this case, each picture element of the image mask may be classified as a foreground region V or a background region H. For example, the image mask may be a binary image representation, wherein foreground region picture elements are assigned the value of 1 or 0 and background region picture elements are assigned the respective remaining value (or vice versa). In this case, the falsifying information components may be identified as picture elements that have the corresponding value for a foreground region picture element.

In a further sub-step, the falsifying information components may be removed and optionally replaced, e.g., by replacement information. Such replacement information may be determined on the basis of the non-falsified information components, for example by interpolation. Alternatively, such replacement information may be determined from topography information generated at an earlier time. If topography information is generated on the basis of image representations, in particular microscopic image representations or image representations of the surroundings, then picture elements or a set of picture elements in particular may be replaced, e.g., the foreground region picture elements mentioned above.

FIG. 3 shows a schematic flowchart of a method according to a further exemplary embodiment of the disclosure. In contrast to the exemplary embodiment depicted in FIG. 1, pieces of topography information TI(t-1), TI(t-k) generated at different times t-1, . . . , t-k are fused in the provision step S2, wherein this fused topography information TI then forms the topography information TI used to generate the augmented image representation AA. In this case, t-1 and t-k denote earlier times at which pieces of topography information TI(t-1), TI(t-k) were generated, where k is a counter variable for the generation steps. For example, these may be retrieved from a storage device 6 of the medical visualization system 4, wherein these pieces of topography information TI(t-1), TI(t-k) are stored therein post generation, in particular in a manner assigned to the corresponding time t-1, t-k.

It is possible that the topography information T(t) in the provision step S2 is generated for a current time or in a current generation step t and taken into account in the fusion. For example, the fusion may be implemented by virtue of a sliding mean value of the pieces of topography information TI(t), TI(t-1), TI(t-k) generated at different times being generated, wherein this mean value than forms the fused topography information.

It is also possible that different sections of the surface 22 that are represented by the fused topography information TI are represented by pieces of topography information TI(t), TI(t-1), TI(t-k) that were generated at different times t, t-1, t-k.

In particular, the pieces of topography information TI(t), TI(t-1), TI(t-k) that were generated at different times t, t-1, t-k may be combined by stitching to form the fused topography information. In this case, stitching may be performed in such a way that the surface sections represented by the pieces of topography information TI(t), TI(t-1), TI(t-k) that were generated at different times t, t-1, t-k are joined seamlessly and, in the totality thereof, represent the entire surface 22 in the detection region of the device for generating topography information TI.

It is also possible that—as explained above—falsifying information components are identified in the topography information TI(t) that was generated at the current time t. In that case, these information components may be replaced by information components of pieces of topography information TI(t-1), TI(t-k) that were generated at an earlier time t-1, t-k, wherein these pieces may represent the part of the surface 22 concealed by the foreign object.

FIG. 4 shows a schematic flowchart of a method according to a further exemplary embodiment of the disclosure. In contrast to the exemplary embodiment depicted in FIG. 1, the final generation of the augmented image representation AA is preceded by a preview image representation VA for the augmented image representation AA being generated and being displayed on a display device, e.g., the display device 20, in a preview display step S3d, the augmented image representation AA being determined finally when a preview-specific confirmation signal is generated. The reception of this confirmation signal is checked in a checking step S3e. If this confirmation signal is not received, especially not within a predetermined period of time, then the generation of the augmented image representation AA may be terminated. However, if it is generated, then the augmented image representation AA is generated, especially as the previously generated preview image representation VA. Alternatively, it is also possible that the checking step S3e includes a check as to whether a rejection signal is received, especially within a predetermined period. If the latter is not received, the augmented image representation AA is generated, especially as the previously generated preview image representation VA. However, if this rejection signal is received, the generation of the augmented image representation AA may be terminated.

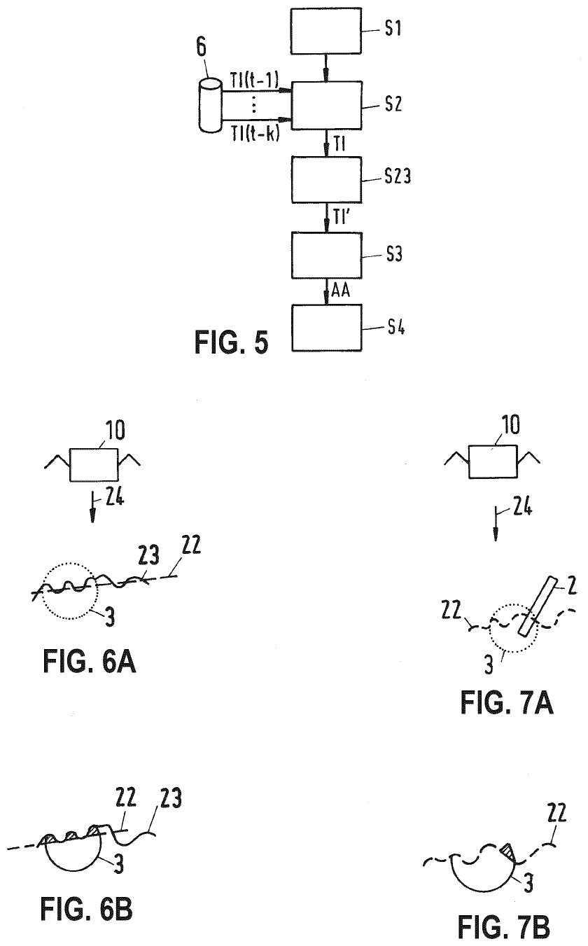

FIG. 5 shows a schematic flowchart of a method according to a further exemplary embodiment of the disclosure. In contrast to the exemplary embodiment depicted in FIG. 1, the topography information TI provided in the provision step S2 is modified dynamically, wherein the augmented image representation AA is generated on the basis of topography information TI′ modified in this way. For example, the topography information TI provided at a first time in the provision step S2 may be used in a first generation step S3 for generating the augmented image representation AA. Then, modified topography information TI′ may be generated in a later generation step S3 carried out at a later time, said generation depending in particular on the time difference between the later time and the first time and on change properties that were determined in advance, for example by evaluating the changes over time between the pieces of topography information TI(t-1), TI(t-k) generated at earlier times. This modified topography information TI′ may be determined in a processing step S23.

FIG. 6A shows a schematic view of an examination region 1 with a surface 22 to be captured and a surgical microscope 10. In this case, an actual spatial profile of the surface 22 to be captured is depicted using dashed lines. An imprecisely detected profile of the surface is represented by a solid line 23. Such an imprecise profile may arise, e.g., as a result of what are known as flickering artifacts, wherein such artifacts may arise, e.g., as a result of a high-frequency fluctuation of distance values from a device that is configured as distance detection device and serves for generating topography information TI (see FIG. 1). A (virtual) tumor object 3, which, e.g., is based on data acquired preoperatively and which may be overlaid on a microscopic image representation by augmentation, is also depicted. Parts of the tumor object 3 located above the actual surface 22 have already been removed. FIG. 6B visualizes an incorrect augmentation that arises due to the imprecisely detected profile. Ideally, only the portion of the tumor object 3 that is situated behind a surface 22 of the examination region 1 in the observation direction 24 should be used by way of the clipping, as explained above, for the purpose of generating the augmented image representation AA. However, on account of the imprecise profile, parts of the tumor object 3 that have already been removed are also displayed in the augmented image representation AA. These parts are represented by the hatched regions in FIG. 6B. Such an incorrect augmentation may be avoided by the fusion, as explained above, of pieces of topography information TI(t), TI(t-1), TI(t-k) generated at different times t, t-1, t-k, or the fusion may at least reduce an extent of the error of this incorrect augmentation.

FIG. 7A shows a schematic view of an examination region 1 with a surface to be captured 22, for the purpose of visualizing a further cause of an incorrect augmentation. In this case, an actual spatial profile of the surface 22 to be captured is depicted using dashed lines. A (virtual) tumor object 3, which, e.g., is based on data acquired preoperatively and which may be overlaid on a microscopic image representation by augmentation, and an instrument 2 are also depicted.

If a spatial profile of the surface 22 is detected in the illustrated scenario, then a part of the section of the instrument 2 that is arranged in front of the surface 22 in the observation direction 24 may incorrectly be detected as part of the surface 22. This instrument 2 thus forms an object that is not part of the surface 22 to be detected but is nevertheless reproduced in an information component of the topography information TI. This information component may be a falsified information component.

FIG. 7B visualizes an incorrect augmentation that arises due to the imprecisely detected profile. Ideally, only the portion of the tumor object 3 that is situated behind the surface 22 in the observation direction 24 should be used by way of the clipping, as explained above, for the purpose of generating the augmented image representation AA. However, on account of the imprecise profile, parts of the tumor object 3 that have already been removed are also displayed in the augmented image representation AA. This is depicted by the hatched regions. These incorrectly represented parts can be traced back to the imprecise detection of the spatial profile of the surface 22 on account of the instrument 2. Such an incorrect augmentation may be avoided by the processing of the topography information TI, as explained above, for reducing falsifying information, or the processing at least reduces an extent of the error of this incorrect augmentation.

FIG. 8 shows a schematic block diagram of a medical visualization system 4 according to the an exemplary embodiment of the disclosure and an examination region 1. The illustration also includes an instrument 2 and a tumor object 3, which are arranged in a detection region of a surgical microscope 10 of the medical visualization system 4.

The tumor object 3 may be a concealed object.

In this case, optional elements of the medical visualization system 4 are depicted using dashed lines. The medical visualization system 4 includes at least one image capture device 5 for microscopic imaging of the examination region 1. The image capture device 5 may be part of the surgical microscope 10 that may include an objective 19 and an end glass. In turn, this surgical microscope 10 may take the form of a stereo surgical microscope, wherein the latter includes a further image capture device 5b for microscopic imaging of the examination region 1, and the image capture devices 5, 5b form a stereo system. A storage device 6, in which additional information ZI may be stored, is also depicted. The medical visualization system 4 also includes an evaluation device 7, which is capable of receiving and evaluating an image representation A1 generated by the image capture device 5, wherein the image representation A1 may be transferred as an image signal to the evaluation device 7 via an interface 21. Using the evaluation device 7, it is possible in particular to perform the steps explained in relation to FIGS. 1 to 5, more particularly the reception step S1, the provision step S2 and the generation step S3.

It is also shown that the medical visualization system 4 may include a device 8 for determining depth information. For example, the latter may take the form of or include a distance sensor. An interface 9 of the medical visualization system 4 for data transmission to further systems, in particular superordinate systems, is also depicted.

Furthermore, the medical visualization system 4 may include:
at least one white-light illumination device 11,
at least one infrared illumination device 12,
at least one fluorescence illumination device 13 for exciting fluorescence,
at least one fluorescence detection device 14 for detecting fluorescence,
at least one surrounding view camera 15,
at least one device 16 for identifying the viewing direction of an observer,
at least one tracking system 17,
at least one input device 18 for operating or controlling the medical visualization system, and
at least one display device 20.

In this case, the elements of the medical visualization system 4 may be data connected and/or signal connected.

Beam filters for providing excitation radiation at wavelengths from a broad spectrum, e.g., the spectrum of the white-light illumination device, or for filtering radiation from a broader spectrum are not depicted here.

The surrounding view camera 15 may be a constituent part of a further tracking system that serves for optical determination of a pose of instruments in the detection region of the surrounding view camera 15. The determination of the pose may be a monoscopic determination. In particular, the determination may also be a marker-based determination.

### LIST OF REFERENCE NUMERALS

1 Examination region
2 Instrument
3 Tumor object
4 Medical visualization system
5 Image capture device of a surgical microscope
5b Further image capture device of the surgical microscope
6 Storage device
7 Evaluation device
8 Device for determining depth information
9 Interface
10 Surgical microscope
11 White-light illumination device
12 Infrared illumination device
13 Fluorescence illumination device
14 Fluorescence detection device
15 Surrounding view camera
16 Device for identifying the viewing direction
17 Tracking system
18 Input device
19 Objective
20 Display device
21 Interface
22 Surface
23 Imprecisely captured surface
24 Observation direction
AA Augmented image representation
VA Preview image representation
TI Topography information
TI′ Modified topography information
t Time
t-1 Time
t-k Time
S1 Reception step
S2 Provision step
S3 Generation step
S3a First sub-step
S3b Second sub-step
S3c Further sub-step
S3d Preview display step
S3e Checking step
S4 Display step
S23 Processing step

What is claimed is:

1. A method for generating an augmented image representation by a medical visualization system, the medical visualization system including at least one image capture device configured for microscopic imaging of an examination region, the method comprising:
receiving a trigger signal;
providing topography information when the trigger signal has been received, the topography information representing a surface to be captured, wherein the surface to be captured is at least partially arranged in the examination region; and
generating the augmented image representation by overlaying additional information on at least one section of a microscopic image representation while taking the topography information into account.

2. The method as claimed in claim 1, wherein the topography information is generated and provided when the trigger signal is received, or
wherein the topography information is provided from already generated topography information.

3. The method as claimed in claim 1, wherein the topography information is generated by evaluating:
microscopic image representations that are generated by the at least one image capture device for microscopic imaging in the medical visualization system, and/or
output signals from a distance measuring device.

4. The method as claimed in claim 1, wherein the trigger signal is generated when at least one of:
a trigger movement is detected,
an input device is operated,
a predetermined change is detected in microscopic image representations and/or in image representations of surroundings, and
a predetermined change is detected in the topography information.

5. The method as claimed in claim 1, wherein the topography information is provided when the trigger signal is received, and/or the topography information is provided to detect a predetermined change, by virtue of at least one of:
generated topography information being processed to reduce falsifying information, and
topography information generated at different times being fused.

6. The method as claimed in claim 5, wherein:
fused topography information is smoothed topography information, or
different sections of the surface represented by the fused topography information are represented by topography information generated at different times.

7. The method as claimed in claim 6, wherein the fused topography information is generated by stitching the topography information generated at different times.

8. The method as claimed in claim 6, wherein falsifying information is replaced by topography information generated at a different time.

9. The method as claimed in claim 5, wherein falsifying information represents at least one of:
an object that is not part of the surface to be captured, and
a foreground region of an image representation of the surface to be captured.

10. The method as claimed in claim 1, wherein the additional information represents at least one object to be augmented, with only those portions of the object to be augmented that are arranged spatially behind a surface of the examination region in an observation direction of the medical visualization system being used for generating the augmented image representation, and/or
wherein an object represented by the additional information is represented in a manner depending on a distance between the object and the surface.

11. The method as claimed in claim 10, wherein the portions used to generate the augmented image representation are generated by cropping the object to be augmented.

12. The method as claimed in claim 1, wherein additional object-specific information is generated based on the topography information, and
wherein the augmented image representation is generated such that the additional object-specific information is represented by the augmented image representation in a manner assigned to the object or object portion that is used to generate the augmented image representation.

13. The method as claimed in claim 1, wherein before finally generating the augmented image representation a preview of the augmented image representation is generated and displayed on a display device, and
wherein the augmented image representation is determined finally when a preview-specific confirmation signal is generated.

14. The method as claimed in claim 1, wherein the topography information provided is modified dynamically, and
wherein the augmented image representation is generated depending on the topography information which is modified dynamically.

15. A medical visualization system, comprising:
at least one image capture device for microscopic imaging of an examination region,
at least one interface for receiving a trigger signal; and
at least one evaluation device, wherein the medical visualization system is configured to perform the method as claimed in claim 1.

16. The method as claimed in claim 3, wherein the distance measuring device is at least one of a TOF and lidar device.

17. The method as claimed in claim 5, wherein fused topography information is topography information smoothed over time.
